(19) United States
(12) Patent Application Publication
(10) Pub. No.: US 2024/0326279 A1
(43) Pub. Date: Oct. 3, 2024

(54) Electrically Driven Sterile Tubing Weld Opener

(71) Applicant: Terumo BCT, Inc., Lakewood, CO (US)

(72) Inventors: Dennis BEBIÉ, Denver, CO (US); Connor UTECH, Fort Collins, CO (US); Lucas HIGGINS, Fort Collins, CO (US); Maxime HARLAN, Parker, CO (US)

(21) Appl. No.: 18/605,958

(22) Filed: Mar. 15, 2024

Related U.S. Application Data
(60) Provisional application No. 63/455,889, filed on Mar. 30, 2023.

(51) Int. Cl. B26F 3/00; A61M 39/08

(52) U.S. Cl. CPC B26F 3/00; A61M 39/08

(57) ABSTRACT

A tube opening device may include a mounting base defining a tube-receiving cavity, a first tube-engaging surface extending into the tube-receiving cavity, a first linear-motion actuator secured to the mounting base and configured to move the first tube-engaging surface between the first retracted position and a first extended position, a second tube-engaging surface extending into the tube-receiving cavity, a second linear-motion actuator secured to the mounting base and configured to move the second tube-engaging surface between the second retracted position and a second extended position, a third tube-engaging surface extending into the tube-receiving cavity, and a third linear-motion actuator secured to the mounting base and configured to move the third tube-engaging surface between the third retracted position and a third extended position.

### CROSS-REFERENCE TO RELATED APPLICATIONS

This application claims the benefit of U.S. Provisional Application No. 63/455,889 filed Mar. 30, 2023. The entire disclosure of the above application is incorporated herein by reference.

### FIELD

The present disclosure relates to systems and assemblies for opening welded tubes, such as tubes welded using tube welding machines.

### BACKGROUND

This section provides background information related to the present disclosure which is not necessarily prior art.

Tube welding machines (also referred to as tube welding devices) are used for connecting closed end tubes which are often connected to bags or similar containers carrying, for example, blood or blood components. Tube welding devices commonly include first and second tube-holding assemblies (e.g., first and second clamps) configured to receive first and second tubes and a space between the first and second tube-holding assemblies configured to receive a wafer (e.g., heated blade), where the tubing welding device is configured to send energy to the space such that the wafer can be heated to desired temperatures. A process used in conjunction with a tubing welding device may include sending energy to the wafer to heat the wafer to a desired temperature and urging the heated wafer into contact with each tube held by the tube-holding assemblies to temporarily seal together opposing surfaces of the respective tubes and creating molten tube ends. At least a portion of one or both of the first and second tube-holding assemblies may then be moved to align and join together the molten tube ends of the first and second tubes. The joint may be cooled and subjected to a stress to open the temporary seals providing fluid communication between the as-joined first and second tubes. Often the temporary seals are broken and the lumen opened by a technician pinching and rolling and/or squeezing and/or pulling the tubing near the joint. These manual processes are often inefficient and may cause the technician to experience hand stress and fatigue, and in certain instances, repetitive motion disorders, like carpal tunnel syndrome, trigger finger, and/or tenosynovitis. Accordingly, it would be desirable to develop systems that reduce these stressors, and methods of using the same.

### SUMMARY

This section provides a general summary of the disclosure and is not a comprehensive disclosure of its full scope or all of its features.

In various aspects, the present disclosure provides an example tube opening device.

In at least one example embodiment, the tube opening device provides a tube-receiving cavity, a tube-engaging surface that extends into the tube-receiving cavity, and an actuator configured to move the tube-engaging surface between a retracted position and an extended position, where a first amount of the tube-engaging surface extends into the tube-receiving cavity in the retracted position, a second amount of the tube-engaging surface extends into the tube-receiving cavity in the extended position, and the second amount is greater than the first amount.

In at least one example embodiment, the tube-engaging surface may have a c-shape, and a concave portion of the c-shape may face the tube-receiving cavity.

In at least one example embodiment, the tube opening device may further include an arm that connects the tube-engaging surface and the actuator.

In at least one example embodiment, the actuator may be a linear-motion actuator.

In at least one example embodiment, the actuator may be coupled to a mounting base and the tube opening device may further include a lid that is movable relative to the mounting base between an open position and a closed position, where in the open position the tube-receiving cavity is accessible and in the closed position access to the tube-receiving cavity is blocked.

In at least one example embodiment, the lid may be configured to apply a pressure to a tube received by the tube-receiving cavity.

In at least one example embodiment, the tube-engaging surface may be a first tube-engaging surface, the actuator may be a first actuator, the retracted position may be a first retracted position, the extended position may be a first extended position, and the tube opening device may further include a second tube-engaging surface that extends into the tube-receiving cavity and a second actuator configured to move the second tube-engaging surface between a second retracted position and a second extended position, where a first amount of the second tube-engaging surface extends into the tube-receiving cavity in the second retracted position, a second amount of the second tube-engaging surface extends into the tube-receiving cavity in the second extended position, the second amount is greater than the first amount, and the second retracted and extended positions are independent from the first retracted and extended positions.

In at least one example embodiment, the second tube-engaging surface may have a c-shape, and a concave portion of the c-shape may face the tube-receiving cavity.

In at least one example embodiment, the tube opening device may further include an arm that connects the second tube-engaging surface and the second actuator.

In at least one example embodiment, the second actuator may be a linear-motion actuator.

In at least one example embodiment, the tube opening device may further include a third tube-engaging surface that extends into the tube-receiving cavity and a third actuator configured to move the third tube-engaging surface between a third retracted position and a third extended position, where a first amount of the third tube-engaging surface extends into the tube-receiving cavity in the third retracted position, a second amount of the third tube-engaging surface extends into the tube-receiving cavity in the third extended position, the second amount is greater than the first amount, and the third retracted and extended positions are independent from the first retracted and extended positions and the second retracted and extended positions.

In at least one example embodiment, the third tube-engaging surface may have a c-shape, and a concave portion of the c-shape may face the tube-receiving cavity.

In at least one example embodiment, the tube opening device may further include an arm that connects the third tube-engaging surface and the third actuator.

In at least one example embodiment, the third actuator may be a linear-motion actuator.

In various aspects, the present disclosure provides another tube opening device.

In at least one example embodiment, the tube opening device may include a mounting base defining a tube-receiving cavity, a first tube-engaging surface extending into the tube-receiving cavity, a first linear-motion actuator secured to the mounting base and configured to move the first tube-engaging surface between the first retracted position and a first extended position, a second tube-engaging surface extending into the tube-receiving cavity, a second linear-motion actuator secured to the mounting base and configured to move the second tube-engaging surface between the second retracted position and a second extended position, a third tube-engaging surface extending into the tube-receiving cavity, and a third linear-motion actuator secured to the mounting base and configured to move the third tube-engaging surface between the third retracted position and a third extended position.

In at least one example embodiment, at least one of the first tube-engaging surface, the second tube-engaging surface, and the third tube-engaging surface may have a c-shape where a concave portion of the c-shape faces the tube-receiving cavity.

In at least one example embodiment, the tube opening device may further include a first arm that connects the first tube-engaging surface and the first linear-motion actuator, a second arm that connects the second tube-engaging surface and the second linear-motion actuator, and a third arm that connects the third tube-engaging surface and the third linear-motion actuator.

In at least one example embodiment, the tube opening device may further include a lid that is movable relative to the mounting base between an open position and a closed position, where in the open position the tube-receiving cavity is accessible and in the closed position access to the tube-receiving cavity is blocked.

In at least one example embodiment, the lid may be configured to apply a pressure to a tube received by the tube-receiving cavity.

In at least one example embodiment, the second linear-motion actuator may be moved independently of the first linear-motion actuator and the third linear-motion actuator is moved independently of the second linear-motion actuator.

### DRAWINGS

The drawings described herein are for illustrative purposes only of selected embodiments and not all possible implementations and are not intended to limit the scope of the present disclosure.

FIG. 1A is a perspective view of an example tube welding device including a first tube-holding assembly and a second tube-holding assembly and having a tube opening device attached thereto in accordance with at least one example embodiment of the present disclosure;

FIG. 1B is a perspective view of the example tube welding device illustrated in FIG. 1A where the first and second tube-holding assemblies are in an open position in accordance with at least one example embodiment of the present disclosure;

FIG. 1C is a perspective view of the example tube welding device of FIG. 1B where the first and second tube-holding assemblies each receive a portion of a first tube and a portion of a second tube in accordance with at least one example embodiment of the present disclosure;

FIG. 1D is a simplified illustration of the movement of the first and second tube-holding assemblies as illustrated in FIGS. 1A-1C in accordance with at least one example embodiment of the present disclosure;

FIG. 2 is a cross-sectional view of a jointed tube, for example, as formed using a tube welding machine (for example, like the example tube welding machine illustrated in FIGS. 1A-1D) in accordance with at least one example embodiment of the present disclosure; and

FIG. 3 is a cross-sectional view of an example tube opening device (for example, as can be using with the example tube welding machine illustrated in FIGS. 1A-1D), which includes three actuators configured to work in tandem to open a welded tube in accordance with at least one example embodiment of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general-purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

Example embodiments will now be described more fully with reference to the accompanying drawings.

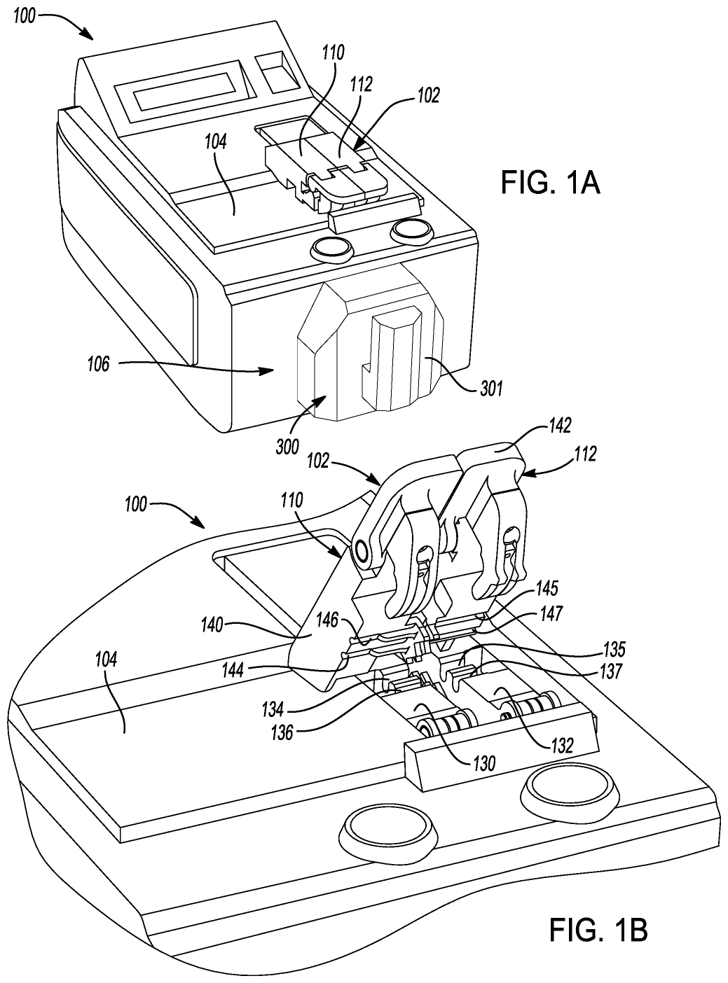

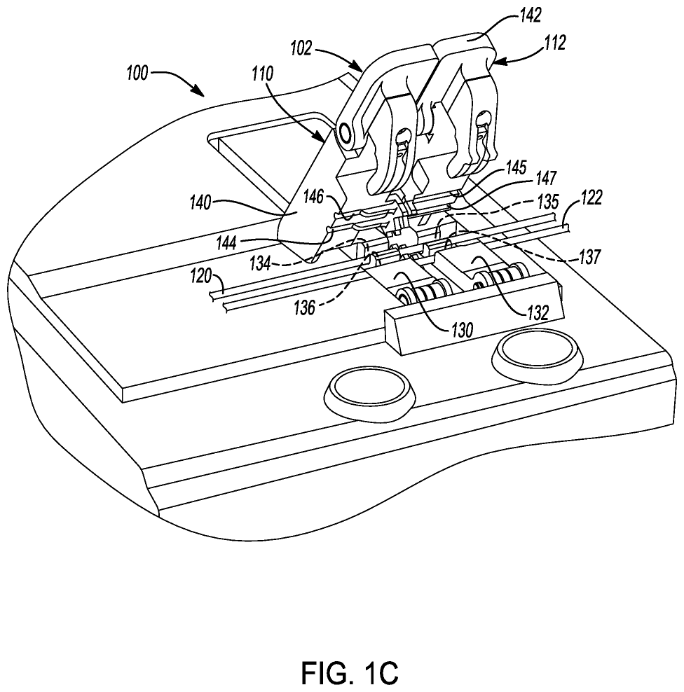

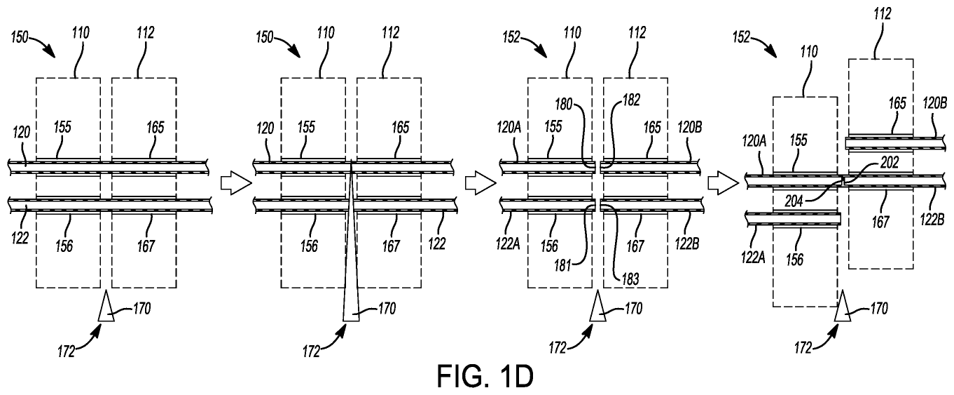

An example tube welding device (also referred to as a tube-joining machine) 100 is illustrated in FIGS. 1A-1D. The tube welding device 100 includes a tube clamp arrangement 102 that is configured to receive at least a portion of a first tube 120 and at least a portion of a second tube 122. The first and second tubes 120, 122 may be connected to bags or similar containers carrying, for example, blood or blood components. In at least one example embodiment, the tube clamp arrangement 102 includes a first tube-holding assembly (which may also be referred to as a first tube-receiving assembly) 110 adjacent to a second tube-holding assembly (also may also be referred to as a second tube-receiving assembly) 112. In at least one example embodiment, the first tube-holding assembly 110 is a first tube-holding clamp, and the second tube-holding assembly 112 is a second tube-holding clamp.

In at least one example embodiment, the first tube-holding assembly 110 may include a first or bottom clamp portion (which may also be referred to as a bottom portion) 130 and a second or top clamp portion (which may also be referred to as a top portion) 140. The bottom clamp portion 130 of the first tube-holding assembly 110 may be movably coupled to the top clamp portion 140 of the first tube-holding assembly 110, such that the first tube-holding assembly 110 may move between a closed position to an open position via the coupling. In at least one example embodiment, the bottom clamp portion 130 of the first tube-holding assembly 110 may be fixedly secured to a major plane 104 of the tube welding device 100 and a top clamp portion 140 may be movable between a first (or closed) clamp state as illustrated, for example, in FIG. 1A and a second (or open) clamp state as illustrated, for example, in FIG. 1B. In at least one example embodiment, the bottom clamp portion 130 of the first tube-holding assembly 110 may be hingedly coupled to the top clamp portion 140 of the first tube-holding assembly 110.

Similarly, in at least one example embodiment, the second tube-holding assembly 112 may include a first or bottom clamp portion (which may also be referred to as a bottom portion) 132 and a second or top clamp portion (which may also be referred to as a top portion) 142. The bottom clamp portion 132 of the second tube-holding assembly 112 may be movably coupled to the top clamp portion 142 of the second tube-holding assembly 112, such that the second tube-holding assembly 112 may move between a closed position to an open position via the coupling. For example, the bottom clamp portion 132 of the second tube-holding assembly 112 may be fixedly secured to a major surface 104 of the tube welding device 100 and a top clamp portion 142 may be movable between a first (or closed) clamp state as illustrated in FIG. 1A and a second (or open) clamp state as illustrated in FIG. 1B. In at least one example embodiment, the bottom clamp portion 132 of the second tube-holding assembly 112 may be hingedly coupled to the top clamp portion 142 of the second tube-holding assembly 112

The first and second tube-holding assemblies 110, 112 are each configured to receive at least a portion of first and second tubes 120, 122. In at least one example embodiment, for example as illustrated in FIGS. 1B and 1C, the bottom clamp portion 130 of the first tube-holding assembly 110 may include a first tube-receiving crevice or recess or surface 134 configured to receive or engage a first portion of the first tube 120 and a second tube-receiving crevice or recess or surface 136 configured to receive or engage a first portion of the second tube 122. Similarly, the top clamp portion 140 of the first tube-holding assembly 110 may include a first tube-receiving crevice or recess or surface 144 also configured to receive or engage the first portion of the first tube 120 and a second tube-receiving crevice or recess or surface 146 also configured to receive or engage the first portion of the second tube 122. For example, the first tube-receiving crevice 134 of the bottom clamp portion 130 of the first tube-holding assembly 110 may align or correspond with the first tube-receiving crevice 144 of the top clamp portion 140 of the first tube-holding assembly 110 to form a first tube-receiving cavity 155 (when the first tube-holding assembly 110 is in a closed state, as illustrated, for example, in FIG. 1A). Likewise, the second tube-receiving crevice 136 of the bottom clamp portion 130 of the first tube-holding assembly 110 may align or correspond with the second tube-receiving crevice 146 of the bottom clamp portion 130 of the first tube-holding assembly 110 to form a second tube-receiving cavity 156 (when the first tube-holding assembly 110 is in a closed state, as illustrated, for example, in FIG. 1A).

Like the bottom clamp portion 130 of the first tube-holding assembly 110, the bottom clamp portion 132 of the second tube-holding assembly 112 may include a first tube-receiving crevice or recess or surface 135 configured to receive or engage a second portion of the first tube 120 and a second tube-receiving crevice or recess or surface 137 configured to receive or engage a second portion of the second tube 122. Like the top clamp portion 140 of the first tube-holding assembly 110, the top clamp portion 142 of the second tube-holding assembly 112 may include a first tube-receiving crevice or recess or surface 145 also configured to receive or engage the second portion of the first tube 120 and a second tube-receiving crevice or recess or surface 147 also configured to receive or engage the second portion of the second tube 122. For example, the first tube-receiving crevice 135 of the bottom clamp portion 132 of the second tube-holding assembly 112 may align or correspond with the first tube-receiving crevice 145 of the top clamp portion 142 of the second tube-holding assembly 112 to form a third tube-receiving cavity 165 (when the second tube-holding assembly 112 is in a closed state, as illustrated, for example, in FIG. 1A). Likewise, the second tube-receiving crevice 137 of the bottom clamp portion 132 of the second tube-holding assembly 112 may align or correspond with the second tube-receiving crevice 147 of the top clamp portion 142 of the second tube-holding assembly 112 to form a fourth tube-receiving cavity 167 (when the second tube-holding assembly 112 is in a closed state, as illustrated, for example, in FIG. 1A).

The tube welding device 100 may be configured to move one or more portions of the first tube-holding assembly 110 and/or one or more portions of the second tube-holding assembly 112. In at least one example embodiment, the tube welding device 100 may include one or more motorized mechanisms for moving the one or more portions of the first tube-holding assembly 110 and/or one or more portions of the second tube-holding assembly 112. For example, as detailed in the Atty. Docket No. 18955-000205-US-PS1, titled AUTOMATIC WELD/RESET MOTION OF CLAMPS AFTER CLAMP CLOSING/OPENING AND TUBE REMOVAL and listing James Ladtkow as inventor, filed the same day hereto and assigned U.S. App. No. 63/455,873, the entire contents of which are herein incorporated by reference.

In at least one example embodiment, the one or more portions of the first tube-holding assembly 110 and/or the one or more portions of the second tube-holding assembly 112 may be movable in a first direction along a major axis of the tube welding machine 100 and/or a second direction along a minor axis of the tube welding machine 100. In at least one example embodiment, for example as illustrated FIG. 1D, the one or more portions of the first tube-holding assembly 110 and one or more portions of the second tube-holding assembly 112 may both be in a first or initial position 150 such that the first tube-receiving cavity 155 of the first tube-holding assembly 110 is aligned with the third tube-receiving cavity 165 of the second tube-holding assembly 112 and the second tube-receiving cavity 156 of the first tube-holding assembly 110 is aligned with the fourth tube-receiving cavity 167 of the second tube-holding assembly 112.

In the initial position 150, the first tube-receiving cavity 155 of the first tube-holding assembly 110 and the third tube-receiving cavity 165 of the second tube-holding assembly 112 aligned therewith may receive adjoining portions of the first tube 120, and the second tube-receiving cavity 156 of the first tube-holding assembly 110 and the fourth tube-receiving cavity 167 of the second tube-holding assembly 112 aligned therewith may receive adjoining portions of the second tube 122, as illustrated for example, in FIGS. 1C and 1D. In at least one example embodiment, the one or more portions of the first tube-holding assembly 110 may be movable relative to the one or more portions of the second tube-holding assembly 112 and/or the one or more portions of the second tube-holding assembly 112 may be movable relative to the one or more portions of the first tube-holding assembly 114, such that, as illustrated in FIG. 1D, a second position 152 is defined, where the first tube-receiving cavity 155 of the first tube-holding assembly 110 is aligned with the third tube-receiving cavity 165 of the second tube-holding assembly 112.

In at least one example embodiment, the tube welding device 100 is configured to receive a wafer 170 within a gap or space between the first tube-holding assembly 110 and the second tube-holding assembly 112. In at least one example embodiment, for example, as illustrated best in FIG. 1D, when the first tube-holding assembly 110 and the second tube-holding assembly 112 are aligned in the initial position 150, the tube welding device 100 may be configured to send energy to the wafer 170 to heat the wafer to a desired temperature (i.e., a tube-joining temperature) and to move the heated wafer 170 from a first non-contact position 172 to a second contact position 174 where the wafer 170 contacts the first and second tubes 120, 122 to temporarily seal together opposing surface of the respective tubes 120, 122 while splitting the tubes into first and second halves 120A, 120B, 122A, 122B and creating molten tube ends (including, for example, first and second molten tube ends 180, 182 for the first tube 120 and first and second molten tube ends 181, 183 for the second tube 122). For example, the heated wafer 170 may cause the portions of the tubes 120, 122 contacted to melt. In at least one example embodiment, the tube-joining temperature may be determined as detailed in the Atty. Docket No. 18955-000211-US-PS1, titled AUTOMATIC POWER ADJUSTMENTS BASED ON TUBING STATE DETECTION FOR TUBE WELDING DEVICES and listing Jesse Janzen, Jeremy Kolenbrander, and James R. Ladtkow as inventors, filed the same day hereto and assigned U.S. App. No. 63/455,817, the entire contents of which are herein incorporated by reference.

In at least one example embodiment, the wafer 170 may be heated using a radiant or conductive heat source the is configured to heat the wafer 170 based on instructions received form a controller. Alternatively, or additionally, the wafer 170 may include an embedded resistive heating element (not shown) and the tube-joining device 100 may be configured to supply an electric current to the embedded resistive heating element based on an instruction received from the controller. In at least one example embodiment, the wafer 170 includes a conductive material, like copper. In at least one example embodiment, the wafer 170 may be a replaceable wafer. For example, the wafer 170 may be removed and replaced after selected use.

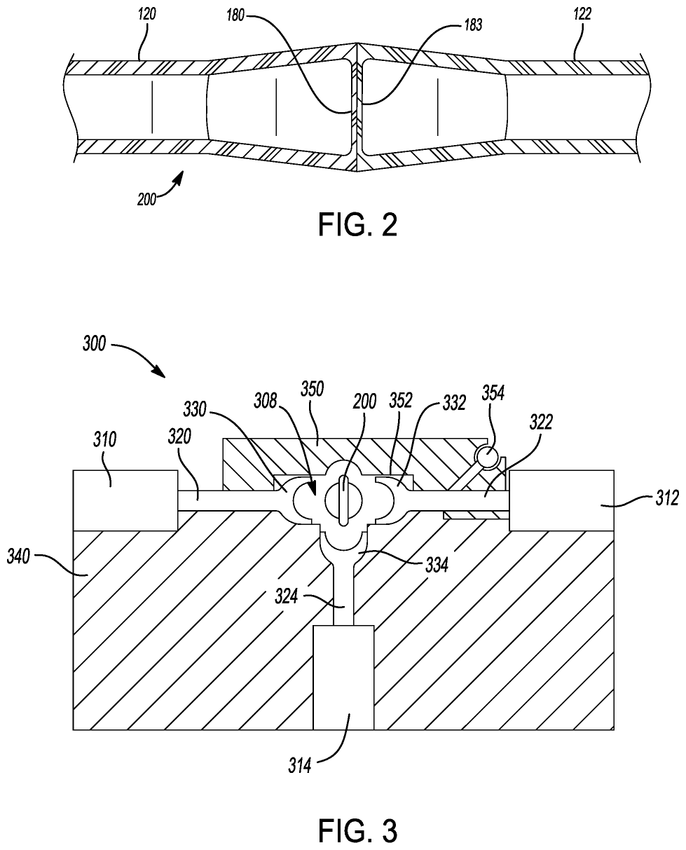

In at least one example embodiment, the tube welding device 100 may include an actuator that is in communication with the controller and that is configured to move the wafer 170 between the first non-contact position 172 and the second contact position 174. After the heated wafer 170 is contacted to the first and/or second tubes 120, 122 (i.e., after the welding event and formation of the molten tube ends 180, 181, 182, 183), the wafer 170 may be displaced—for example, returned to its first non-contact position 172 (i.e., retracted)—and the tube welding device 100, as discussed above, may be configured to move the one or more portions of the first tube-holding assembly 110 and/or the one or more portions of the second tube-holding assembly 112 to the second position 152 such that the first molten tube end 180 of the first tube 120 is brought into contact with the second molten tube end 183 of the second tube 122 (and/or the second molten tube end 183 of the second tube 122 is brought into contact with the first molten 180 of the first tube 120) to form a jointed tube 200, as illustrated, for example, in FIG. 2. In at least one example embodiment, the jointed tube 200 may be cooled (for example, passively to room temperature, which is often between about 20° C. and about 22° C.) and subjected to a stress that breaks the temporary seals of the molten tube ends 180, 183 opening the lumen 210 providing fluid communication between the as-joined first and second tubes 120, 122.

In at least one example embodiment, the tube welding device 100 may include a tube opening device (also referred to as a tube opener) 300 that is configured to break the temporary seals of the molten tube ends 180, 183 opening the lumen 210 to provide fluid communication between the as-joined first and second tubes 120, 122. For example, as illustrated, in at least one example embodiment, the tube opening device 300 may be fixedly or removably secured to a first side 106 of the tube welding device 100. Although the tube opening device 300 is illustrated as being disposed on a first side 106 of the tube welding device 100, it should be appreciated that, in other example embodiments, the tube opening device 300 may be disposed on one or more other sides of the tube welding device 100 or not attached or coupled to the tube welding device 100.

In at least one example embodiment, for example, as illustrated in FIG. 3, the tube opening device 300 may include three actuators 310, 312, 314 coupled or secured to a mounting base 340, where each of the actuators 310, 312, 314 includes an arm or length 320, 322, 324 that at least partially extends into a tube-receiving cavity or slot 308 and is moveable between a retracted state or position and an extended state or position. The three actuators 310, 312, 314 are in communication with a controller, similar to the wafer 170 as discussed above.

The tube-receiving cavity 308 is configured to receive a jointed or welded tube, like the jointed tube 200 illustrated in FIG. 2. In at least one example embodiment, the tube-opening device 300 may include a lid 350 that is movable relative to the mounting base 340, for example from a first or opened position to a second or closed position via a hinge 354, where in a close position the lid 350 encloses the tube-receiving cavity 308. In at least one example embodiment, an interior-facing surface 352 of the lid 350 may have a configured to apply a pressure to the jointed tube 200 received by the tube-receiving cavity 308. The lid 350 is configured such that the actuators 310, 312, 314 are able to move the arms 320, 322, 324 between the retracted positions and the extended positions when the lid 350 is in the closed position (as illustrated).

In at least one example embodiment, the ends of each of the arms 320, 322, 324 extending into the tube-receiving cavity 308 includes a tube-engaging surface 330, 332, 334 that has at least one surface that stimulates or corresponds with the radius of an open tube formed by breaking the temporary seals of the jointed tube 200. A first tube-engaging surface 330 of the first actuator 310 may be the same as or different from a second tube-engaging surface 332 of the second actuator 312 which may be the same as or different from a third tube-engaging surface 334 of the third actuator 314. For example, in at least one example embodiment, as illustrated, the tube-engaging surfaces 330, 332, 334 may have a general c-shaped where concave portions face the tube-receiving cavity 308. The actuators 310, 312, 314 may be configured to move the tube-engaging surfaces 330, 332, 334 in tandem to apply an opening force or forces on the joining tube 200 to form an opened, continuous tube. In at least one example embodiment, the opening force may be a pinching force. In at least one example embodiment, the actuators 310, 312, 314 may be linear-motion, electrical actuators. The inclusion of three actuators 310, 312, 314 provides greater flexibility in the use of the tube opening device 300. For example, as a result of the three actuators 310, 312, 314 the tube 200 may have any orientation when placed into the tube-receiving cavity 308.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A tube opening device comprising:
a tube-receiving cavity;
a tube-engaging surface that extends into the tube-receiving cavity; and
an actuator configured to move the tube-engaging surface between a retracted position and an extended position, a first amount of the tube-engaging surface extending into the tube-receiving cavity in the retracted position, a second amount of the tube-engaging surface extending into the tube-receiving cavity in the extended position, the second amount being greater than the first amount.

2. The tube opening device of claim 1, wherein the tube-engaging surface has a c-shape, and a concave portion of the c-shape faces the tube-receiving cavity.

3. The tube opening device of claim 1, wherein the tube opening device further includes:
an arm that connects the tube-engaging surface and the actuator.

4. The tube opening device of claim 1, wherein the actuator is a linear-motion actuator.

5. The tube opening device of claim 1, wherein the actuator is coupled to a mounting base and the tube opening device further includes:
a lid that is movable relative to the mounting base between an open position and a closed position, in the open position the tube-receiving cavity is accessible, in the closed position access to the tube-receiving cavity is blocked.

6. The tube opening device of claim 5, wherein the lid is configured to apply a pressure to a tube received by the tube-receiving cavity.

7. The tube opening device of claim 1, wherein the tube-engaging surface is a first tube-engaging surface, the actuator is a first actuator, the retracted position is a first retracted position, the extended position is a first extended position, and the tube opening device further includes:
a second tube-engaging surface that extends into the tube-receiving cavity; and
a second actuator configured to move the second tube-engaging surface between a second retracted position and a second extended position, a first amount of the second tube-engaging surface extending into the tube-receiving cavity in the second retracted position, a second amount of the second tube-engaging surface extending into the tube-receiving cavity in the second extended position, the second amount being greater than the first amount, the second retracted and extended positions being independent from the first retracted and extended positions.

8. The tube opening device of claim 7, wherein the second tube-engaging surface has a c-shape, and a concave portion of the c-shape faces the tube-receiving cavity.

9. The tube opening device of claim 7, wherein the tube opening device further includes:
an arm that connects the second tube-engaging surface and the second actuator.

10. The tube opening device of claim 7, wherein the second actuator is a linear-motion actuator.

11. The tube opening device of claim 7, wherein the tube opening device further include:
a third tube-engaging surface that extends into the tube-receiving cavity; and
a third actuator configured to move the third tube-engaging surface between a third retracted position and a third extended position, a first amount of the third tube-engaging surface extending into the tube-receiving cavity in the third retracted position, a second amount of the third tube-engaging surface extending into the tube-receiving cavity in the third extended position, the second amount being greater than the first amount, the third retracted and extended positions being independent from the first retracted and extended positions and the second retracted and extended positions.

12. The tube opening device of claim 11, wherein the third tube-engaging surface has a c-shape, and a concave portion of the c-shape faces the tube-receiving cavity.

13. The tube opening device of claim 11, wherein the tube opening device further includes:
an arm that connects the third tube-engaging surface and the third actuator.

14. The tube opening device of claim 11, wherein the third actuator is a linear-motion actuator.

15. A tube opening device comprising:
a mounting base defining a tube-receiving cavity;
a first tube-engaging surface extending into the tube-receiving cavity;
a first linear-motion actuator secured to the mounting base and configured to move the first tube-engaging surface between the first retracted position and a first extended position;
a second tube-engaging surface extending into the tube-receiving cavity;
a second linear-motion actuator secured to the mounting base and configured to move the second tube-engaging surface between the second retracted position and a second extended position;
a third tube-engaging surface extending into the tube-receiving cavity; and
a third linear-motion actuator secured to the mounting base and configured to move the third tube-engaging surface between the third retracted position and a third extended position.

16. The tube opening device of claim 15, wherein at least one of the first tube-engaging surface, the second tube-engaging surface, and the third tube-engaging surface has a c-shape and a concave portion of the c-shape faces the tube-receiving cavity.

17. The tube opening device of claim 15, wherein the tube opening device further includes:
a first arm that connects the first tube-engaging surface and the first linear-motion actuator;
a second arm that connects the second tube-engaging surface and the second linear-motion actuator; and
a third arm that connects the third tube-engaging surface and the third linear-motion actuator.

18. The tube opening device of claim 15, wherein the tube opening device further includes:
a lid that is movable relative to the mounting base between an open position and a closed position, in the open position the tube-receiving cavity is accessible, in the closed position access to the tube-receiving cavity is blocked.

19. The tube opening device of claim 18, wherein the lid is configured to apply a pressure to a tube received by the tube-receiving cavity.

20. The tube opening device of claim 15, wherein the second linear-motion actuator is moved independently of the first linear-motion actuator and the third linear-motion actuator is moved independently of the second linear-motion actuator.
